(19) United States
(12) Patent Application Publication
(10) Pub. No.: US 2013/0123565 A1
(43) Pub. Date: May 16, 2013

(54) Quality Assurance Device and Method in Radiotherapy

(71) Applicant: Jean-Marc Denis, Ottignies (BE)

(72) Inventor: Jean-Marc Denis, Ottignies (BE)

(73) Assignee: QRAY SPRL, Court-Saint Etienne (BE)

(21) Appl. No.: 13/635,041

(22) PCT Filed: Mar. 15, 2011
(86) PCT No.: PCT/EP2011/053904

§ 371 Date: Oct. 9, 2012

(30) Foreign Application Priority Data
Mar. 15, 2010 (BE) BE2010/0169

(51) Int. Cl. A61N 5/10

(52) U.S. Cl. CPC A61N 5/1075
USPC 600/1

(57) ABSTRACT

The invention relates to a method of quality assurance of an apparatus for radiotherapy (10) by a photon beam (20) directed toward an object or a patient (30), comprising the following steps: the object or the patient (30) is galvanically isolated from a reference potential; a pico-ammeter (60) is linked between the object or the patient (30) and the reference potential; the photon beam (20) is directed toward the object or the patient (30); the electric charge (Q) arising in the object or the patient (30) and/or the electric current (I) flowing between the object or the patient (30) and the reference potential are/is measured by means of the pico-ammeter (60). The invention also pertains to an apparatus suitable for the execution of this method.

### TECHNICAL DOMAIN

The invention pertains to a method and a device for quality assurance of a photon beam radiotherapy apparatus. It also pertains to a method and an apparatus for measuring the dose and/or the dose rate deposited by a photon beam in a patient or a phantom.

The treatment of cancer by radiotherapy, in particular by a photon beam directed toward a tumor of a patient, is a well known technique. Typically, a beam of electrons with energy of between 4 and 25 MeV produced by a Linac is dispatched to a target X. This produces a photon beam. This photon beam is shaped by means of equalizer filters (flat filters) and collimators and is directed toward a patient. It is also possible to use a gamma radioactive source such as a Cobalt 60 source. During the application of this technique, it is of vital importance that the dose applied to the patient be in accordance with the prescription, both in its geometric distribution and in its intensity. If the dose delivered at the level of the tumor is too low, the probability of checking the tumor is not optimal and gives rise to an increased risk of recurrence. Conversely, too high a dose at the level of the “organs at risk” engenders an increased risk of post-treatment complications. Now, numerous sources of error and of uncertainty may occur and present risks for the patient. This is why various means have been provided making it possible to guard against these risks. These means include, among others, quality assurance, and “in-vivo” measurement.

Quality assurance in radiotherapy is the set of procedures which ensure the consistency of the prescription, and the completely safe achieving of this prescription, as regards the dose deposited in the target volume, as well as a minimum dose in the surrounding healthy tissue. Quality assurance reduces the risk of accidents and errors, but is also aimed at increasing the chances of these errors being detected and corrected as early as possible. The quality assurance programs for a Linac radiotherapy apparatus can comprise daily, monthly, annual tests of various operating parameters of the machine. In a quality assurance program, it is necessary to define a reference: the value of the expected parameters. It is also necessary to define a tolerance threshold: the tolerated discrepancies and the type of intervention to be undertaken if a measurement strays from the tolerance bracket. Finally, it is necessary to define the periodicity of the tests, and the corrective actions to be undertaken. A quality assurance program in radiotherapy is described in “Comprehensive QA for Radiation Oncology: Report Of AAPM Radiation Therapy Committee Taskgroup 40” (Med. Phys. 21 (4), April 1994). Within the framework of these tests, it is possible to measure the distribution in space of the dose deposited by irradiating a “water phantom” in which a detector is positioned at the various measurement points. A “phantom” is a device for measuring dose and radiation. It comprises a proof body and one or more dosimeters placed in or on the proof body. A “water phantom” is a phantom consisting of a vessel filled with water, of parallelepipedal shape. A dosimeter may be moved around within the vessel and makes it possible to reconstruct the 3D distribution of the dose in the water volume. Solid phantoms also exist. They are made of a material, usually polymer, in which diodes or ionization chambers may be placed at appropriate locations or are provided in cubbyholes of the phantom. The solid phantom can consist of a material simulating the shape and absorption characteristics of a human body, including the variations of the these characteristics, for example because of the bony structures.

Document U.S. Pat. No. 3,122,640 discloses a method and an apparatus for measuring the flux of incident photons arising from an X-ray or gamma-ray source. In this apparatus, a scatterer 10 receives the incident photon beam. Compton electrons are produced in this scatterer, mainly in the direction of the incident beam. These Compton electrons are then absorbed by a central electrode 12 and then measured by means of a circuit comprising a voltmeter 25. This apparatus does not make it possible, however, to determine the dose deposited in an arbitrary proof body and still less in a patient. It cannot therefore be used in a method of quality assurance of a radiotherapy apparatus.

The in-vivo tests comprise a measurement of dose during treatment. They may be carried out by means of one or more dosimeters, for example a semiconductor-based detector or a thermoluminescent dosimeter (TLD) placed on the patient's skin, in the field of the beam. By using this technique, the dose or the dose rate is measured at particular points of the irradiated field. Outside of these points, the dose actually delivered remains unknown. It is not therefore possible to detect an error in the geometric distribution of the irradiated field. It is also possible to dispose a two-dimensional (2D) detector between the source and the patient (transmission-based chamber). The geometric distribution of the photon flux is then detected. It is important in this case to have a detector which does not attenuate or disturb the beam, that is to say a “transparent” detector. Other tests may be carried out by placing a film or a 2D detector downstream of the patient. The fluence emerging from the patient is thus measured after having passed through the latter. All treatment machines are equipped with detectors, usually transmission-based chambers, measuring the rate of the ionizing beam in the machine. This measurement is calibrated so as to be able to predict the dose delivered to the patient. Unfortunately, this measurement is made upstream of certain elements modifying the beam before reaching the patient (like the multileaf collimator). An error at the level of these elements will therefore not be seen at the level of the dose monitor. Furthermore, this measurement is made upstream of the patient and does not make it possible to circumvent a patient positioning error.

However, experience has shown that despite quality assurance programs and in-vivo measurements, accidents occur. An inventory of accidents that have occurred, in particular accidents involving patients, can be read in “J M Cosset, P Gourmelon: “Accidents en radiotherapie: un historique” [Accidents in radiotherapy: a log], Cancer/Radiother 6 (2002)”. An article in the “New York Times” of 24 Jan. 2010 describes in detail the circumstances and causes of two accidents that caused the death of patients treated by radiotherapy. The cause of one of the accidents was a computer error. The other originated from the absence of a filter. Incidents or accidents can thus occur following errors of dose or of dose rate, of dimension of the irradiation field (collimator position error), of errors with the energy of the incident beam, of patient position errors (e.g. error in the value of the source-skin distance SSD). There therefore exists a need for a simple and reliable procedure and an apparatus which makes it possible to detect in real time a malfunction of a treatment by radiotherapy.

### SUMMARY OF THE INVENTION

According to a first aspect, the invention relates to a method of quality assurance of an apparatus for radiotherapy by a photon beam directed toward an object or a patient, comprising the following steps: the object or the patient is galvanically isolated from its/his environment; a pico-ammeter is linked between the object or the patient and a reference potential; the photon beam is directed toward the object or the patient; the electric charge arising in the object or the patient (Q) and/or the electric current (I) flowing between the object or the patient and the reference potential and/or the potential difference arising between the object or the patient (30) and the reference potential are/is measured by means of the pico-ammeter. The reference potential may be earth. As explained hereinafter, the charge Q, the current (I) and the potential difference result from the action of the photon beam on the object or the patient. The measurement of the charge may be obtained by using an electrometer or by integrating the current measured by the picoammeter.

It is advantageously possible to compare the measurement of said charge (Q) and/or of said current (I) and/or of said potential difference with an expected value.

It is possible to establish said expected value beforehand by a calculation in accordance with the Monte Carlo method.

It is also possible to establish said expected value beforehand by a prior measurement carried out in accordance with the invention by means of the same object or of a comparable object.

It is, finally, possible to establish said expected value beforehand by calculation of an analytical model in which, on departure of the fluence of photons, of the distribution of matter in the object or the patient, and of the curve of deposition of dose in the matter as a function of depth, the integral is determined of the dose deposited over the extent of the exit surface of the object or of the patient. It is possible to specify the method by including other contributions in the analytical model, especially the electrons generated during the passage of the photon beam within a collimator, and photoelectrons ejected on the face of entry of the beam into the object or the patient.

It is advantageously possible to establish a calibration curve by a theoretical calculation of the total dose and of the charge (Q) and/or of the current (I) and/or of said potential difference which correspond thereto, in accordance with the Monte Carlo method. It is thus possible to determine the total dose by virtue of the measurement of the charge (Q) and/or of the current (I) and/or of the potential difference.

The calibration curve can also be obtained by undertaking the method of the invention simultaneously with the measurement of the dose and/or of the dose rate by means of a dosimeter by means of the same object or of a comparable object.

The calibration curve can, finally, be obtained by calculation of an analytical model in which, on departure of the fluence of photons, of the distribution of matter in the object or the patient, and of the curve of deposition of dose in the matter as a function of depth, the dose and/or rate of dose and of said charge (Q) and/or said current (I) and/or said potential difference which correspond thereto are/is determined.

It is advantageously possible to generate an alert signal if said charge (Q) and/or said current (I) and/or the potential difference differs from the expected value by more than a pre-established tolerance.

According to a second aspect, the invention relates to a device for quality assurance of an apparatus for radiotherapy by a photon beam directed toward object or a patient, comprising the following elements: a holding device for holding the object or the patient, the object or the patient being isolated galvanically from a reference potential; a pico-ammeter able to measure the charge (Q) carried by the object or the patient and/or the current (I) flowing between the object or the patient and the reference potential and/or a voltmeter able to measure the potential difference arising between the object or the patient and the reference potential; an acquisition device able to record said charge and/or said current and/or said potential difference.

Furthermore the device can comprise the following elements: means able to receive an expected value of said charge (Q) and/or of said current (I) and/or of said potential difference; means able to compare the charge (Q) and/or the current (I) and/or said potential difference with the expected values and to generate an alert signal if said charge (Q) and/or said current (I) and/or said potential difference differs from the expected value by more than a pre-established tolerance. It is thus possible to alert the operator in real time if a malfunction occurs.

The holding device can comprise a table on which is disposed an insulating layer, the proof body being disposed on the insulating layer.

The holding device can furthermore comprise a second insulating layer and a conducting layer which are disposed between the table and the proof body, a second pico-ammeter being able to measure the charge (Q′) carried by the conducting layer and/or the current (I′) flowing between the conducting layer and the reference potential and/or a second voltmeter being able to measure the potential difference arising between the object or the patient (30) and the reference potential.

According to a third aspect, the invention relates to a phantom for use in the method or the device of the invention, which is made of an electrically conducting solid material and which comprises a contact electrode.

### BRIEF DESCRIPTION OF THE DRAWINGS

FIG. 1 schematically represents a device in accordance with the invention.

FIG. 2 represents experimental results obtained with this device.

FIG. 3 schematically represents the possible interactions of a photon beam with a proof body.

FIG. 4 schematically represents an embodiment of a device in accordance with the invention.

### DETAILED DESCRIPTION OF THE INVENTION

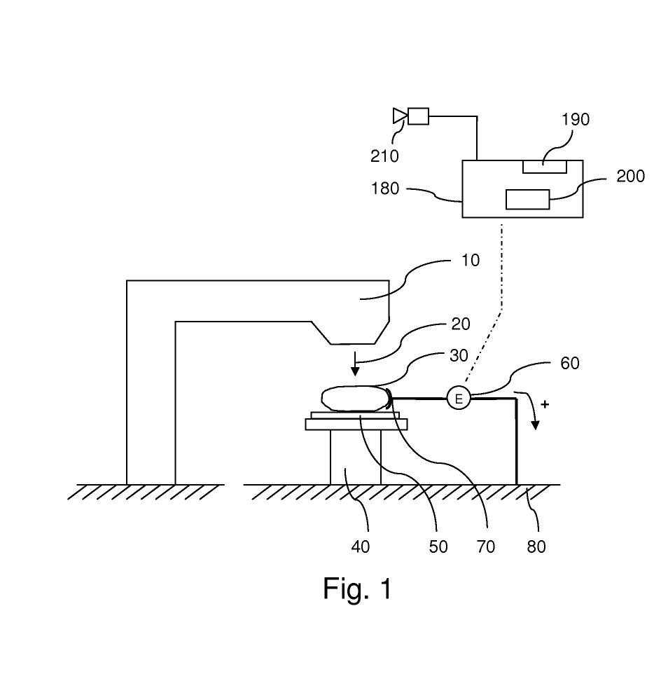

The applicant has observed that, in an unexpected manner, by irradiating a proof body by means of a photon beam, the proof body having previously been placed on a support galvanically isolated from the earth, a measurable charge of the proof body was observed, in conjunction with the dose deposited by the photon beam. The experimental device is represented in FIG. 1. A radiotherapy apparatus 10 (or any other source of photons, for example a Co60 source) emits a photon beam 20 toward a proof body 30. The proof body 30 may be a “water phantom” or an arbitrary volume, in a material exhibiting radiation absorption characteristics similar to those of the human body. It can also be a patient. It must have sufficient conductivity to allow conduction of the current within the proof body. It can also be made of a, for example metallic, conducting material. The proof body 30 is placed on a table 40, and, unlike in the known configurations, an insulant 50 is placed between the proof bodies 30 and the table 40. The insulant may be for example a polymer. It must exhibit a resistance greater than that exhibited by the proof body. Tests have been performed using as insulant 50 a plate of expanded polystyrene foam 3 cm thick. It is also possible to use mylar or any other insulating material.

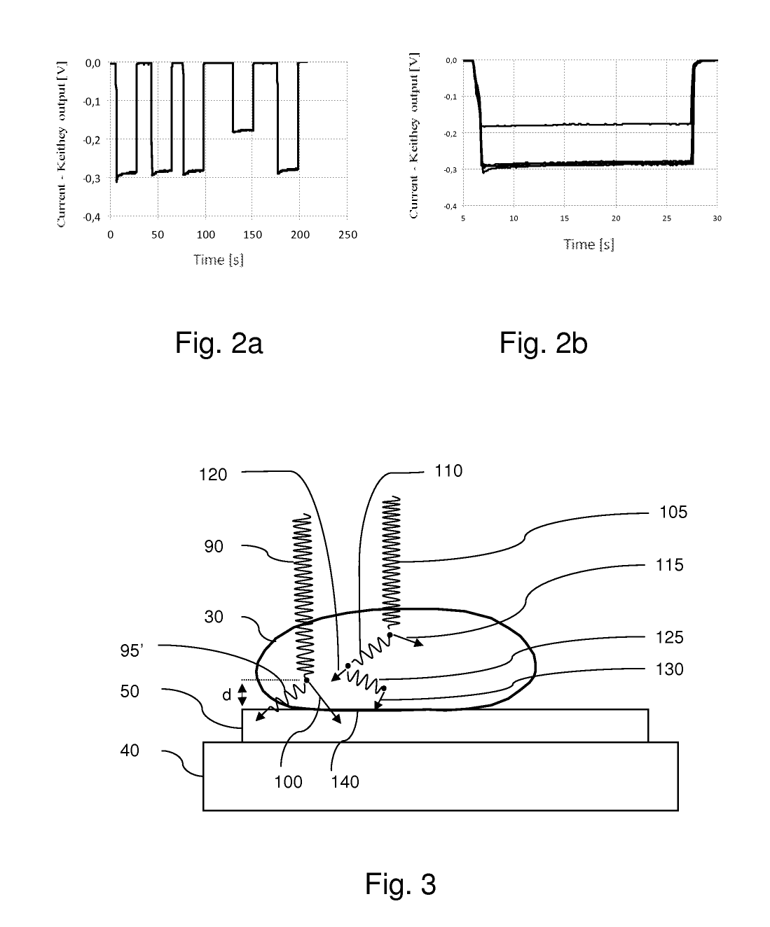

It should be noted that if the table 40 is itself insulating there is no need to add an insulant. An electrometer or pico-ammeter 60 is linked on the one hand to an electrode 70 attached to the proof body, and on the other hand to earth 80. The pico-ammeter 60 makes it possible to measure and display and/or record the current and/or the charge as a function of time. It is also possible to use a voltmeter and to measure the potential difference between the proof body. FIG. 2a represents the measurement of the current during the irradiation of a proof body with a photon beam of an intensity of 2 Gy/min (dose rate under protocol reference conditions), obtained by Bremsstrahlung of electrons of 6 MeV delivered over a field of 10 cm×10 cm measured at the surface of entry of the beam into the proof body. The proof body used is a plexiglass pan filled with water up to a height of 20 cm. Irradiation periods are followed by off periods. A current of about 0.3 nA is observed. It is observed that this current flows from the proof body toward earth. This current contributes to compensating for a deficit of electrons which is engendered by the ejection of electrons out of the proof body. Other phenomena contributing to this current are discussed hereinbelow. During the fourth irradiation period, the irradiation field has been reduced by closing a multileaf collimator. A proportional decrease in the measured current is observed. In FIG. 2b, the five measurements have been reproduced and superimposed, thereby illustrating the perfect reproducibility of the experiment.

The device (represented in FIG. 1) in accordance with the invention can comprise a data acquisition device 180, linked to the pico-ammeter 60. This acquisition device 180 may be a simple personal computer. It can comprise means 190 able to receive an expected value of the charge or of the current. These means may simply be a keyboard and a screen for entering the expected values, or a linking interface for example a DICOM interface with a treatment program or calculation system. The acquisition device can comprise means for comparing the measured value with the expected value, and for generating an alarm signal, for example by means of a klaxon 210 or a luminous signal. The operator is then warned in real time of the occurrence of an error.

These observations may be explained in the light of general knowledge about the interaction of photons and electrons with matter, and the application of this general knowledge to the experimental situation described hereinabove.

Photons passing through matter can deposit their energy by several mechanisms:
Photoelectric effect: the photon interacts with a bound electron of an atom and disappears. This electron termed a “photoelectron” is then ejected from the atom with a kinetic energy equal to the initial energy of the incident photon minus the binding energy of the electron.
Compton effect: when the energy of the photon is substantially greater than the binding energy of the electron, the photon loses part of its energy and ejects an electron. Energy and momentum are conserved in this process. The energy of the scattered photon is less than that of the incident photon and is scattered in a different direction. This photon can undergo several successive Compton scatterings before disappearing through the photoelectric effect. The “recoil” electron also carries off a part of the energy.
Pair creation: the photon disappears and an electron-positron pair is created, the combined kinetic energy of which is equal to the energy of the incident photon minus the mass energy of the two particles created.

In the energy range of the photons used in radiotherapy, it is mainly the Compton effect which occurs, in particular when the matter traversed is of low atomic number Z, as in living matter (H, C, N, O). Whatever the type of interaction mechanism, it is the charged particle (electron or positron) which will actually deposit energy as it journeys through the matter by lineal energy transfer. An electron ends up depositing all its energy and being stopped after journeying a distance in water of the order of 2 mm for 1-MeV electrons of the order of 2 cm for 10-MeV electrons. This distance is called the “stopping distance”. It has then deposited all its energy during its journey.

Two examples of possible interaction diagrams have been represented in FIG. 3. It is known that photons can penetrate deeply into matter. In a first diagram, an incident photon 90 penetrates the proof body 30 and undergoes a Compton interaction producing a scattered photon 95 and a recoil electron 100. This interaction has taken place at a distance d from the exit face 140 of the proof body which is less than the stopping distance of the electron, the distance d being measured in the direction of journey of the electron. The electron therefore leaves the volume of the proof body 30 and can ultimately be deposited in the insulant 50. It can also pass through the insulant and rejoin the earth. It thus contributes to the current that would be measured by the pico-ammeter. In a similar diagram, an electron could also be ejected into the air, through a lateral face of the proof body, or through the photon beam entry face.

In a second diagram, a photon 105 penetrates less deeply into the proof body and undergoes a first Compton interaction producing a scattered photon 110 and a recoil electron 115. This recoil electron stops after journeying within the matter of the proof body 30, during which it deposits all its energy. The scattered photon 110 undergoes a second Compton interaction producing in its turn an electron 120 and a scattered photon 125. The scattered photon 125 then causes an interaction of photoelectric effect type producing a photoelectron 130. This photoelectron 130 may stop within the matter of the proof body, as represented in the figure, or, if it is produced in proximity to the surface of the proof body, be ejected from the latter. In both the first and the second diagram, the ejected electrons may be ejected through the exit face 140, and also through the lateral faces and the entry face. These two exemplary possible journeys show that interaction diagrams exist which, such as the first diagram, eject an electron from the proof body, and others, such as the second diagram, which do not eject any. The photoelectric effect and the creation of pairs may also contribute to the ejection of electrons. The interactions producing the ejection of an electron all occur at a distance from the exit face 140 which is less than the stopping distance of an electron. This distance being short, it is possible to make the approximation that the current is given by the expression:

Iₑ=K∫_{S}D(x,y)·dS

where Iₐ is the measured current, K a proportionality coefficient, D the dose deposited by the photons in proximity to the exit face 140, dS an element of this surface, and the integral is extended to the beam exit surface S. The coefficient K depends on the nature of the materials, and the energy of the incident photon beam.

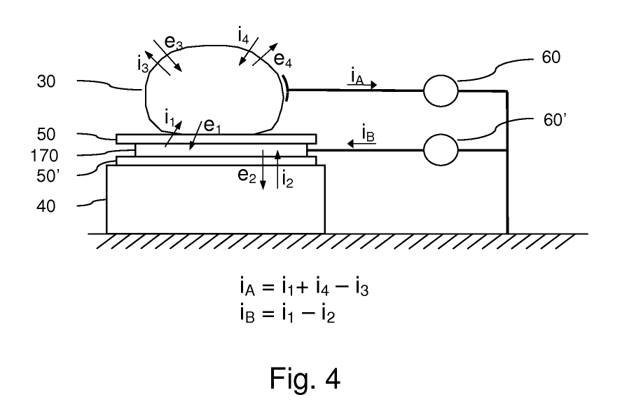

FIG. 4 represents an embodiment of the invention, in which the elements identical to those of FIG. 1 bear the same numbers. Furthermore, in this device, an additional insulating plate 50′ has been disposed between the table 40 and a conducting plate 170, itself placed under the insulating plate 50. A second pico-ammeter 60′ is linked between the conducting plate 170 and the reference potential. Represented in this diagram are the electron fluxes eₓ, and by reverse arrows iₓ the corresponding currents. In this diagram,
e₁ represents the Compton electrons ejected through the beam exit face 140, which were discussed in the previous paragraph and are shown diagrammatically by the arrow 100 in FIG. 3. This is by far the most significant component of the currents involved in this device.
e₂ represents the Compton electrons ejected through the beam exit face from the plate 170.
e₃ represents the electrons emitted by a collimator when it is traversed by a beam.
e₄ represents the electrons emitted “backward” (that is to say in a direction opposite to the incident beam) on the surface of entry of the beam into the proof body 30.

The currents i_{A} and i_{B} measured by the pico-ammeters 60 and 60′ respectively are given by the equations:

i_{A}=i₁−i₃+i₄

i_{B}=i₁−i₂

The device of FIG. 4 therefore makes it possible to analyze and to separate the various components of the measured currents. The chosen thickness of the insulating layer has an impact on the value of i2: the thicker it is, the more the photons which pass through it generate electrons and therefore a significant current i2.

In an old document (Gross B., “The Compton Current”, Zeitschrift für Phyzik, 155, 479-487 (1959)) the author describes that the absorption of photons (X rays or gamma rays) of energy lying between 0.5 and 3 MeV is due mainly to the Compton effect. The author develops a theory, and then describes an experimental device (FIG. 1 of this document) in which a plexiglas collector 1, associated with a block of lead 3, constitute a means for collecting the electrons ejected during the interaction of the incident beam with the plexiglas housing 2. This device does not make it possible, however, to measure the entirety of the charges ejected out of the housing 2, since only those ejected toward the collector 1 and gathered by the latter are measured. Moreover, just as for document U.S. Pat. No. 3,122,640 discussed hereinabove, this device does not make it possible to quantify the dose absorbed by an arbitrary proof body, such as a quality assurance phantom and still less in a patient.

The quality assurance method in accordance with the invention makes it possible, by means of the measurement of the current I(e), of the charge (Q) or of the potential difference, to determine a deviation of one of the following parameters with respect to their setpoint value:
1. the intensity of the beam
2. the energy of the beam
3. the dose rate of the beam and its variation over time (for example in IMRT)
4. the size of the beam
5. the position of the patient
6. the morphology of the patient
7. the equipment traversed by the beam (the table, the immobilization systems) can have an effect on the current measured.

In a method in accordance with the invention, a patient is placed on the table of a radiotherapy apparatus 10 represented in FIG. 4. For a given treatment, the charge accumulated on the patient may be determined. Measurement of the charge therefore makes it possible to verify a possible deviation of one or more of the 7 parameters listed above. It is also possible to measure the electric current directly. This current is of the order of 0.3 nA for a dose rate of 2 Gy/min delivered in a field of 10×10 cm.

The current Ie is measured during treatment and compared with an expected value of this current.

The expected value may have been obtained in various ways:
a) By a calculation in accordance with the Monte Carlo method: A program such as MCNP or Geant is used to carry out a statistical simulation of the possible interactions of a given beam incident on a given geometry of the patient. The number of electrons ejected and therefore the expected current is deduced therefrom. This is the most reliable and the most precise method. However, it requires significant calculation means, and the provision of a definition of the geometry and materials present. Furthermore the calculation program used must contain precise nuclear models. By using this method, it is possible to take account of ancillary aspects which arise when a collimator is used to limit the extent of the photon field. It is known that the result of this collimation is to also create electrons some of which may be captured by the proof body and have an impact on the current measured. The model can therefore take account of the currents i₁, I₂, I₃ and i₄ discussed hereinabove.
b) By a simulation prior to the patient's treatment by applying the treatment to a “phantom” of geometry and make-up close to the patient to be treated. It is also possible to apply scale factors for example as discussed in “The photon-fluence scaling theorem for Compton-scattered radiation” (John S. Pruitt et al. Med. Phys. 9(2) March/April 1982)
c) By comparison with the value of the current Iₑ obtained during an earlier fraction of this patient's treatment.
d) By an analytical model. In an analytical model, the fluence φ is determined. Knowing the distribution of matter and the curve of deposition of dose in the matter as a function of depth, the dose deposited by this fluence φ over the whole of the extent of the exit surface of this flux of photons is determined. This calculation gives the contribution of the current i₁ to the current i_{A} measured. Similar analytical calculations can lead to the values of the currents i₃ and i₄.

In a preferred variant of the invention, it is possible to correlate the value of the measured current Ie or charge Qe with the dose rate or with the total dose deposited by the beam. To this end, it is possible to perform a calibration. The calibration curve may be obtained by a Monte Carlo calculation, by simultaneous measurement of the current Ie or of the charge Qe and of the dose rate or of the dose, by known dosimetry means, or by an analytical calculation such as described hereinabove.

In the present description, the measured currents discussed hereinabove may be time-dependent values. In general, they will vary as a function of the fluence issuing from the radiotherapy apparatus and/or of the position of the collimators which may vary over time. The values of the currents measured as a function of time can constitute a verification of the treatment delivery procedure in the course of which the position of the collimators is varied as a function of time (IMRT). It is thus possible to detect an error in the operation of the collimators.

When the method is undertaken on a patient, the electrical conductivity of the body is sufficient to allow the flow of the currents ix toward the contact electrode 70. To undertake the method using a phantom, it is necessary to have a phantom exhibiting sufficient electrical conductivity. The applicant has therefore designed a range of phantoms in the known geometric or anthropomorphic shapes, but moreover exhibiting sufficient electrical conductivity. These phantoms can consist of a polymer filled with carbon fibers to ensure electrical conductivity. Furthermore, they are furnished with a contact electrode 70 making it possible to link it to a pico-ammeter or a voltmeter.

The device and the method of the invention exhibit numerous advantages:
They provide a very simple, inexpensive and reliable means of detecting in real time a deviation of one or more parameters of the irradiation of a patient.
The measurement device is entirely independent of the radiotherapy apparatus. It can be installed very easily on any existing radiotherapy apparatus.
They are simple to implement (it suffices to place a single electrode anywhere on the patient's skin);
They do not depend on the location at which the electrode is placed;
They allow real-time measurement of the radiation level dispatched to the patient;
the measurement of the radiation level does not depend on the exterior conditions (pressure, temperature, etc.);
the measurement makes it possible to detect a deviation at the level of the) the dose, of the dose rate, of the energy of the beam, of the type of beam (e- or photon), of the position of the patient, of the source-skin distance (SSD), of the orientation of the gantry, of the position of the MLC, etc.

In the method and the device of the invention, it is the patient or the proof body (phantom) which constitutes the sensor. The identity between the two gives the method great reliability: any source of error, for example as regards the position or the nature of a sensor, is eliminated. It suffices that this sensor has sufficient conductivity to allow the pico-ammeter to measure the current or the charge, or the voltmeter to measure the potential difference, this being the case for the body of a patient. The point of connection of the measurement apparatus to the patient may be chosen freely as a function of convenience and may be for example be a conducting bracelet surrounding the patient's wrist or ankle, away from the irradiated part.

The terms and descriptions used here are proposed by way of illustration only and do not constitute limitations. The measurement of the charge Q, of the current I or of the potential difference are means among others of measuring the number of electrons ejected out of the object or of the patient minus the number of electrons received by the latter. The person skilled in the art will recognize that numerous variations are possible in the spirit and the scope of the invention such as described in the claims which follow and their equivalents. In said claims, all the terms should be understood in their widest acceptation unless indicated otherwise.

1. A method of quality assurance of an apparatus for radiotherapy (10) by a photon beam (20) directed toward object or a patient (30), comprising the following steps:
the object or the patient (30) is galvanically isolated from its/his environment;
a pico-ammeter and/or a voltmeter (60) are/is linked between the object or the patient (30) and a reference potential;
the photon beam (20) is directed toward the object or the patient (30);
one determines
by means of the pico-ammeter (60) the electric charge (Q) arising in the object or the patient (30) and/or the electric current (I) flowing between the object or the patient (30) and the reference potential
and/or by means of the voltmeter (60) the potential difference arising between the object or the patient (30) and the reference potential.

2. The method as claimed in claim 1, wherein, the measurement of said charge (Q) and/or of said current (I) and/or of said potential difference are/is compared with an expected value.

3. The method as claimed in claim 2, wherein said expected value is established beforehand by a calculation in accordance with the Monte Carlo method.

4. The method as claimed in claim 2, wherein said expected value is established beforehand by a prior measurement carried out in accordance with claim 1 by means of the same object (30) or of a comparable object.

5. The method as claimed in claim 2, wherein said expected value is established beforehand by calculation of an analytical model in which, on departure of the fluence of photons, of the distribution of matter in the object or the patient, and of the curve of deposition of dose in the matter as a function of depth, the integral is determined of the dose deposited over the extent of the exit surface (140) of the object or patient (30).

6. The method as claimed in claim 1, wherein a calibration curve is established beforehand by a theoretical calculation of the total dose and of the charge (Q) and/or of the current (I) and/or of the potential difference which correspond thereto, in accordance with the Monte Carlo method.

7. The method as claimed in claim 1, wherein a calibration curve giving the dose and/or the dose rate is established beforehand as a function of said charge (Q) and/or of said current (I) and/or of said potential difference by undertaking the method of claim 1 simultaneously with the measurement of the dose and/or of the dose rate by means of a dosimeter by means of the same object (30) or of a comparable object.

8. The method as claimed in claim 1, wherein a calibration curve is established beforehand by calculation of an analytical model in which, on departure of the fluence of photons, of the distribution of matter in the object or the patient, and of the curve of deposition of dose in the matter as a function of depth, the dose and/or rate of dose and of said charge (Q) and/or said current (I) and/or said potential difference which correspond thereto are/is determined.

9. The method as claimed in claim 2, wherein an alert signal is generated if said charge (Q) and/or said current (I) and/or said potential difference differs from the expected value by more than a pre-established tolerance

10. A device for quality assurance of an apparatus for radiotherapy (10) by a photon beam (20) directed toward object or a patient (30), comprising the following elements:
a holding device (40) for holding the object or the patient (30), the object or the patient (30) being isolated galvanically from its environment;
a pico-ammeter (60) able to determine the charge (Q) carried by the object or the patient and/or the current (I) flowing between the object or the patient and a reference potential and/or a voltmeter (60) able to measure the potential difference arising between the object or the patient (30) and the reference potential;
an acquisition device (180) able to record said charge and/or said current and/or said potential difference.

11. The device as claimed in claim 10, further comprising the following elements:
means (190) able to receive (190) an expected value of said charge (Q) and/or of said current (I) and/or of said potential difference;
means able to compare (200) the charge (Q) and/or the current (I) and/or said potential difference with the expected values and means able to generate an alert signal (210) if said charge (Q) and/or said current (I) and/or said potential difference differs from the expected value by more than a pre-established tolerance.

12. The device as claimed in claim 10, wherein the holding device (40) comprises a table (40) on which is disposed an insulating layer (50), the proof body (30) being disposed on the insulating layer (50).

13. The device as claimed in claim 12, wherein the holding device (40) furthermore comprises a second insulating layer (50′) and a conducting layer (170) which are disposed between the table (40) and the proof body (30), a second pico-ammeter and/or voltmeter (60′) being able to measure the charge (Q′) carried by the conducting layer (170) and/or the current (I′) flowing between the conducting layer (170) and the reference potential and/or the potential difference arising between the object or the patient (30) and the reference potential.

14. A phantom for use in the methods of claim 1, wherein it is made of an electrically conducting solid material and comprises a contact electrode (70).
